(19) United States
(12) Patent Application Publication
(10) Pub. No.: US 2023/0196563 A1
(43) Pub. Date: Jun. 22, 2023

(54) MAGNETIC RESONANCE IMAGE PROCESSING METHOD

(71) Applicant: UCL Business Ltd, London (GB)

(72) Inventors: Shonit Punwani, London (GB); David Atkinson, London (GB); Yangcan Gong, London (GB); William Devine, London (GB); Daniel Crawford Alexander, London (GB)

(21) Appl. No.: 17/996,245

(22) PCT Filed: Apr. 16, 2021
(86) PCT No.: PCT/GB2021/050911

§ 371 Date: Oct. 14, 2022

(30) Foreign Application Priority Data
Apr. 17, 2020 (GB) 2005630.5

(51) Int. Cl. G06T 7/00; G01R 33/50; G06T 7/13; A61B 5/055

(52) U.S. Cl. CPC G06T 7/0012; G01R 33/50; G06T 7/13; A61B 5/055; G06T 2207/10088; G06T 2207/30081; G06T 2207/30068

(57) ABSTRACT

An image processing method comprising: receiving MRI data representing a scan of an organ of a patient, the MRI data including multiecho data for a plurality of pixels; for each of a plurality of pixels of the MRI data: fitting the multiecho data to a simulated decay curve; calculating a tissue index based on at least one parameter of the simulated decay curve; and comparing the tissue index to a threshold to determine a tissue type; wherein each pixel of the multiecho data consists of 16 or fewer echoes.

### CROSS-REFERENCE TO RELATED APPLICATIONS

This application is a 371 National Phase Entry of International Patent Application No. PCT/GB2021/050911 filed on Apr. 16, 2021, which claims the benefit of British Patent Application No. 2005630.5 filed on Apr. 17, 2020, the contents of which are incorporated herein by reference in their entirety.

### FIELD OF THE INVENTION

The present invention relates to image processing, in particular to processing MRI images in order to discriminate between tissue types.

### BACKGROUND OF THE INVENTION

Magnetic resonance imaging (MRI) is often used in the diagnosis of cancer in various organs. One technique, proposed for use in detecting cancerous lesions in the prostate, uses a multiecho spin-echo sequence to examine the luminal water fraction (LWF). The luminal water fraction is the ratio of glandular water to cellular/extracellular water and is determined on a pixel-by-pixel basis across multiple image slices through the patient's prostate. Regions of low LWF are indicative of tumor. [Ref 1] and [Ref 2] have proposed T₂ MRI sequences using 64 echoes or 32 echoes to determine the LWF. A large number of echoes is required to make an accurate determination of LWF; however, the imaging procedure is therefore slow, limiting the number of slices that can be obtained and/or the number of patients that can be imaged in a given time.

### SUMMARY OF THE INVENTION

It is an aim of the present invention to provide an improved method of identifying disease areas, for example clinically significant tumors, in the prostate or other organs of the human or animal body.

According to an embodiment of the invention, there is provided an image processing method comprising: receiving MRI data representing a scan of an organ of a patient, the MRI data including multiecho data for a plurality of pixels; for each of a plurality of pixels of the MRI data: fitting the multiecho data to a simulated decay curve; calculating a tissue index based on at least one parameter of the simulated decay curve; and comparing the tissue index to a threshold to determine a tissue type; wherein each pixel of the multiecho data consists of 16 or fewer echoes.

According to an embodiment of the invention there is also provided a method of imaging comprising: performing a magnetic resonance imaging process to obtain multiecho MRI data corresponding to a scan of an organ of a patient; and processing the multiecho MRI data using the method described above.

The present invention also provides apparatus such as MRI scanners, computer systems, and computer programs for implementing the above method.

Therefore, the present invention can enable detection of potential tumors in organs using a quicker imaging technique, allowing an increase in resolution and/or a quicker imaging process. Embodiments of the invention can also be used to distinguish between clinically significant and nonsignificant tumors.

The present invention is applicable for prostate cancer screening. Currently there is no UK screening program for prostate cancer. Previously, levels of blood prostate specific antigen (PSA) have been evaluated, but PSA has not been adopted as a screening test as there are many false positives and to a lesser extent false negative results. The present invention could enable prostate screening.

The present invention is also applicable to reducing unnecessary biopsies in men with an elevated PSA. Multiparametric MRI (mpMRI) is now standard of care for men with an elevated PSA. However, approximately 50% of men that undergo mpMRI followed by biopsy do not have significant cancer. Replacing mpMRI with this method would reduce the number of false positive studies and thereby reduce the number of biopsies.

The present invention is also applicable to reduce the time/cost of MRI by replacing a 35-45-minute MRI study with a 5-10-minute alternative.

### BRIEF DESCRIPTION OF THE FIGURES

Exemplary embodiments of the invention will now be described with reference to and as illustrated in the accompanying drawings, in which:

FIG. 1 is a schematic drawing of an environment in which the present invention may be implemented;

FIG. 2 is a flowchart of a method of an embodiment;

FIGS. 3-5 are examples of H&E stained histology sections of pancreatic tissue with Gleason 4+4 lesions (FIG. 3), Gleason 3+4 lesions (FIG. 4) and normal tissue in PZ (FIG. 5) and their corresponding T₂ echo distributions;

FIG. 6 is a graph showing median and interquartile range of LI of lesions containing nonsignificant/benign biopsy findings and significant cancer (n=142);

FIG. 7 is a graph showing ROC analysis of LI for separation of patients with and without significant cancer (n=142), with the solid line signifying performance of LI (ROC-AUC 0.89) and the dotted line illustrating the line of identity (ROC-AUC 0.5);

FIG. 8 is a graph showing median and interquartile range of LI of lesions for Likert 3 and 4 cases; and

FIG. 9 is a graph indicating Bland—Altman analysis of LI values.

### DETAILED DESCRIPTION OF THE INVENTION

The present invention provides a new imaging technique to determine a new parameter, referred to herein as luminal index (LI), using multiecho T₂-weighted imaging. A scan according to the new method can be performed quickly, e.g., in less than 10 minutes, and in initial work (initially on approximately 82 patients, subsequently a further 31 patients, with biopsy) demonstrates a better ability to characterize lesions than mpMRI alone.

The new imaging technique (referred to herein as LI-MRI) may therefore improve the detection of prostate cancer after a PSA test, replacing mpMRI, improving diagnosis at a lower cost. The new imaging technique may also be used for primary screening, replacing both PSA and mpMRI and enabling prostate cancer screening.

If the new imaging technique is used as a replacement for mpMRI, in a selective or screening context, the scan results can be processed, either as part of the scanner software or within a cloud-based solution, to generate an LI-MRI map. The LI-MRI map together with one of the T₂-weighted images used to generate the map can be reviewed by the radiologist to score individual regions/lesions on a 1-5 Likert scale for suspicion of significant prostate cancer. A targeted biopsy to confirm tumor can be performed on those regions that score higher than a selected Likert threshold (for example, either 3 or 4).

The new imaging technique can also be used with patients with a known diagnosis of cancer to monitor evolution of the lesion with time as the LI value is correlated with Gleason grade of tumor. This allows yearly scans to be performed with patients on active surveillance and for the lesion volume and LI value to be used as a combined index of stability/progression.

Because LI-MRI is a short sequence without significant risk of artefact, it is ideal for deployment as a screening tool. A short, e.g., 5-minute, scan can be performed in men based on age (e.g., 50-75). This can generate a screen positive or screen negative results based on quantitative and/or qualitative evaluation of the LI-MRI images either by radiologist or by software.

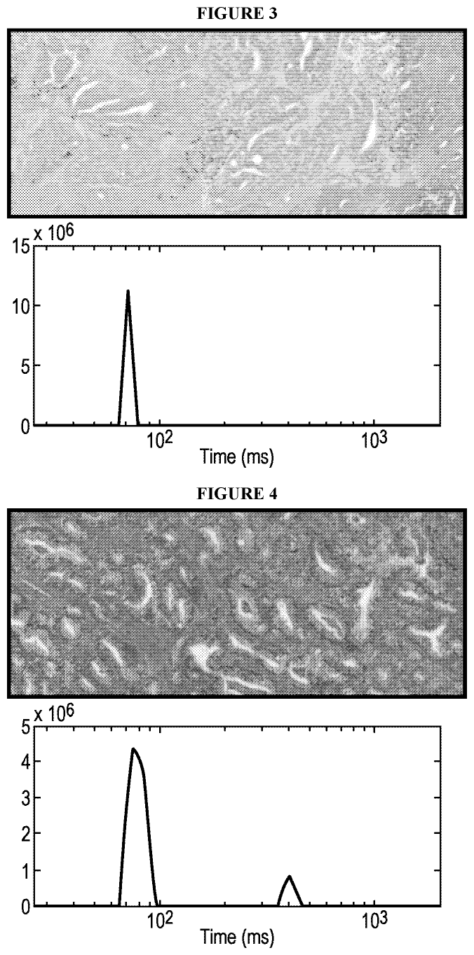

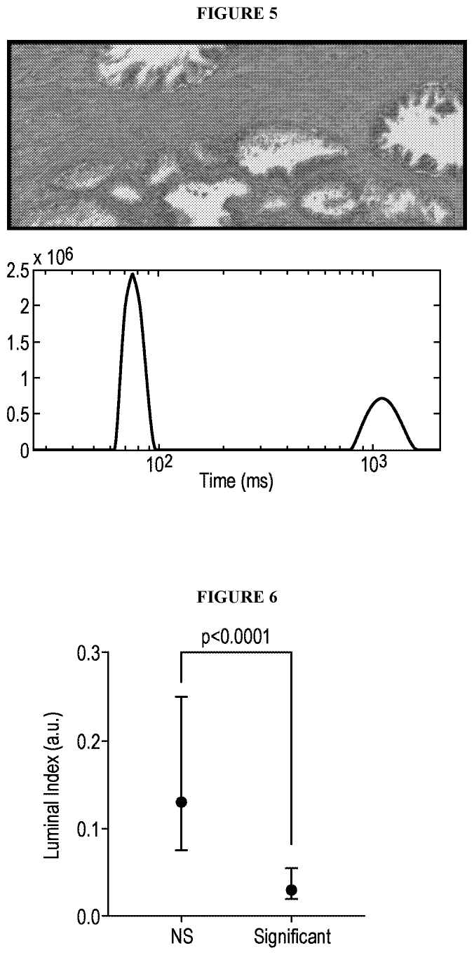

FIGS. 3-5 illustrate the principle on which the present invention is based. These figures are examples of H&E stained histology sections of pancreatic tissue with Gleason 4+4 lesions (FIG. 3), Gleason 3+4 lesions (FIG. 4) and normal tissue in the peripheral zone (FIG. 5) and their corresponding T₂ echo distributions. It can be seen that with increasing Gleason grade, there is less lumen space in histology sections, also a decreasing area under the long T₂ peak (note change in scale of the y-axis) [Ref 4].

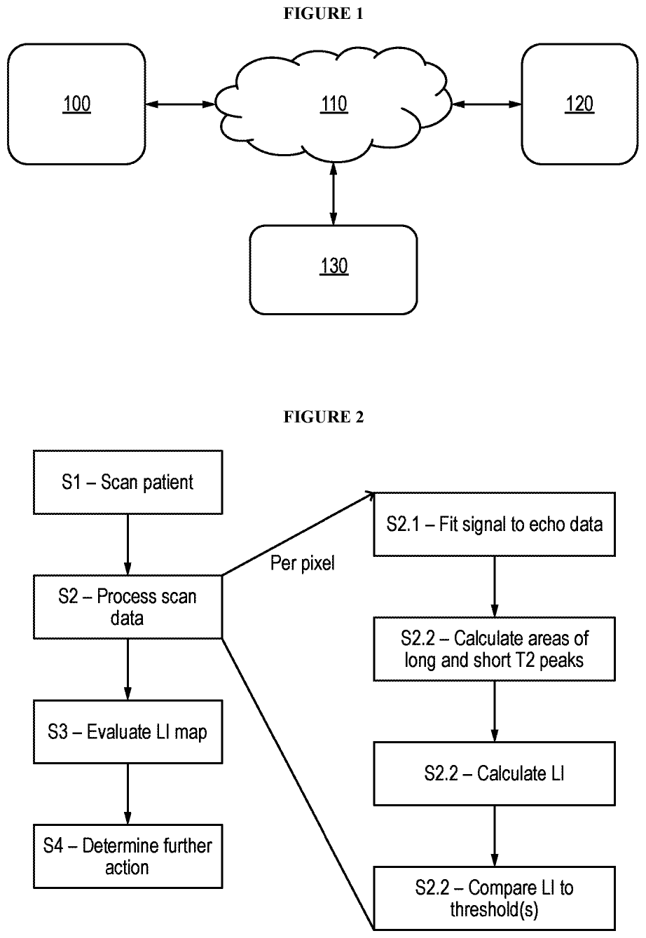

FIG. 1 depicts an environment in which the present invention may be put into practice. An MRI scanner (100) is capable of performing a conventional multiecho T₂ scan. A variety of MRI scanners capable of performing such a scan are available. MRI scanner (100) may be connected, via network (110), to an image processor (120) and a user workstation (130). Image processor (120) is configured to process images provided by MRI scanner (100) as described below and may be embedded in MRI scanner (100) or formed by one or more independent computer systems. User workstation (130) is configured to control MRI scanner (100) and/or review outputs of image processor (120). User workstation 130 may also be combined with either or both of MRI scanner (100) and image processor (120) or may be an independent computer system or a thin client.

A flowchart of a method according to an embodiment of the invention is shown in FIG. 2. First the relevant organ, e.g., prostate, of the patient is scanned (S1) using MRI scanner (100) and the output data transferred to the image processor (120) to be processed (S2) on a pixel-by-pixel basis into an LI map as discussed below. The LI map is evaluated (S3) to enable a determination (S4) of further action. The determination might be to refer the patient for a biopsy, in which case the LI map can be used to select the location of the biopsy. The determination may be that the patient should be monitored, in which case the LI map may form a reference against which future scan results are compared. The determination might be that no further action is required.

Scanning step (S1) can be performed on any suitable MRI scanner capable of acquiring multiple echo and multiple slice T₂ imaging. In an embodiment, the scanning step is performed to image a plurality of parallel planes of the organ to provide a 3D LI map, so that the pixels of each image may be considered voxels, representing a volume of the organ. In an embodiment scanning step (S1) is performed so as to obtain for each pixel an echo train comprising 16 or fewer echoes, desirably 8 echoes or 6 echoes. It is possible to use different echo spacing, for example first a few echoes with shorter TE followed by echoes with longer TE. Desirably the total period of echoes is at least 500 ms to provide enough information in order to determine T_{long} and Tₛₕₒᵣₜ distributions.

In prior art methods, an echo train of 32 or 64 echoes has been considered essential to achieve an accurate determination of luminal water fraction (LWF). However, the present inventors have determined that fewer echoes can be used to determine a new measure, referred to herein as luminal index (LI), that does not directly measure the luminal water fraction but nevertheless adequately distinguishes between normal tissue and tumor.

The processing of scan data (S2) to derive LI values is performed on a pixel-by-pixel basis. They can be derived for the whole region scanned or limited to a contour of the organ under investigation. In some cases it might be sufficient to process only a sample of pixels.

For each pixel, a simulated echo signal is fitted to the echo data (S2.1). A variety of algorithms for fitting to the echo data can be used, for example a regularized nonnegative least squares (NNLS) algorithm to fit a multiexponential model or a model with two Gaussian distributions fitted by least squares regression. The simulated echo signal is used to calculate the areas under the long and short T₂ peaks, which gives an indication of the relative amounts of water in the luminal compartments and stroma and epithelia compartment, respectively.

In an embodiment, the luminal index LI is calculated as the area under long T₂ distribution (A_{L}) divided by the sum of area under short (A_{S}) and long T₂ distribution, i.e.:

$$LI = \frac{{A}_{L}}{{A}_{L} + {A}_{S}} \qquad ( Eq . 1 )$$

Alternatively or in addition, the luminal index can be calculated on the basis of one or more of the following parameters: A_{L}; ratio A_{L}:A_{S}; Tₛₕₒᵣₜ; T_{long};

$$\frac{{A}_{S}}{{A}_{S} + {A}_{L}} ;$$

and the magnitude ratio between the two peaks (α).

A threshold value LIₜ can be determined such that values of LI below LIₜ indicate tumor and values above LIₜ indicate normal tissue. In some cases, it is desirable to use two thresholds: an LI value below a first threshold LIₜ₁ indicates tumor and an LI value above a second threshold LIₜ₂ (LIₜ₁<LIₜ₂) indicates normal tissue whilst LI values between the two thresholds are indeterminate. In a visualization of an LI map colors can be used to indicate different tissue types, e.g., green for normal, yellow for indeterminate and red for tumor. A continuous color range can be used with, for example, a red-green color scale mapped to a range of LI values. Alternatively or in addition, the LI values can be displayed as a contour map.

In experiments, the LI value was found to vary from measurement to measurement by up to ±80% but the difference between significant and nonsignificant findings is approximately 400%. Therefore, thresholds as described above can be used to achieve an accurate distinction between normal tissue and tumor in spite of measurement variation. By using fewer echoes than known techniques, the MRI scanning step can be performed much more quickly and/or with a larger number of slices (better volume resolution) reducing costs and/or increasing accuracy of the detection of tumor.

The absolute values of the thresholds may depend on the manner of calculation of the luminal index (which may be dimensionless and/or have arbitrary units), the MRI scanner and program used and in particular the number of echoes on which the calculation of LI is based. Thresholds may be determined empirically, based on scans of known healthy organs and organs known to have tumor. Given thresholds determined for a specific scanner type and/or imaging protocol, thresholds for other scanners and/or other imaging protocols can be determined using calibration scans of imaging phantoms. First and second (e.g., lower and upper) thresholds can be derived from a single ROC curve threshold value set by the 95% limits of agreement from studies determining repeatability.

Absolute values of threshold may also depend on the organ being investigated and or different parts of the organ. For example, different thresholds may be applied in peripheral and central parts of the prostate. In an embodiment, a threshold for use in the transition zone of the prostate is ⅓ of the threshold used in the peripheral zone.

### EXAMPLES

Experiments were conducted to validate the techniques described above. Luminal Water data was acquired on a subcohort of another study. Patient inclusion criteria were: (1) men referred for prostate mpMRI following previous biopsy more than 6 months earlier and (2) biopsy naive men presenting a clinical suspicion of prostate cancer. Patient exclusion criteria included (1) men unable to have an MRI scan, or in whom artefact would reduce the quality of the MRI, (2) men unable to give informed consent, (3) previous treatment (prostatectomy, radiotherapy, brachytherapy) of prostate cancer, (4) ongoing hormonal treatment for prostate cancer, and (5) previous biopsy within 6 months of scheduled mpMRI [Ref 1].

Biopsy cohort inclusion criteria are: (1) patients have an mpMRI score equal to or greater than Likert score 3; (2) Patient has targeted biopsy; (3) Luminal water scan has a matching slice with mpMRI and the top score MR lesion was biopsied.

Following informed consent, 108 patients initially and then a further 49 were scanned on a 3.0T scanner (Philips Achieva; Philips Medical Systems, Best, the Netherlands) using a 32-channel cardiac coil. A multiecho spin-echo sequence was used. The MR parameters are listed in Table 1 below. All men underwent a standard mpMRI examination as part of routine investigation of elevated prostate specific antigen. The mpMRI was reported by a board-certified radiologist.

TABLE 1

| Parameter | 32-echo |
|---|---|
| Number of echoes | 32 |
| TE (ms) | 31.25 |
| TR (ms) | 8,956 |
| Acquisition voxel size (mm³) | 2 × 2 × 4 |
| FOV (mm³) | 180 × 180 × 26 |
| Scan Duration (mm:ss) | 05:49 |

88 patients initially and subsequently a further 40 underwent targeted biopsy of suspicious lesions and the contralateral prostate. Following biopsy, an experienced radiologist, aware of their positive and negative biopsy status, contoured max MR score lesion on T₂-weighted images. A matching lesion in luminal water scan is then drawn by the radiologist in a single slice on the third echo (93.75 ms) which is a similar echo time to a traditional axial T₂-weighted prostate image (˜100 ms). Two of the initial cases and four of the subsequent cases were excluded due having a biopsy date later than six months after the scan. Four initial cases and 4 of the subsequent cases were excluded because there was no matching slice in luminal water scan and mpMRI scan. One case has technical issue and therefore also excluded. A total of 82 regions of interest (ROI) were contoured across the initial patient cohort and a further 31 across the subsequent cohort, with a maximum of one biopsy positive or one biopsy negative lesion per patient.

A cohort of 20 Likert score 2 patients from the initial cohort and 9 from the subsequent cohort was randomly selected from the bigger study. Radiologists drew an ROI on a peripheral zone MR benign region on T₂-weighted images and then transferred to the matching luminal water scan slice with adjustments if needed. These 20 Likert score 2 cases were treated as biopsy benign cases as mpMRI has approximately 90% sensitivity in detecting prostate cancer using a 1.5T scanner [Ref 2, 3]. MRI parameters are listed below.

To assess repeatability of luminal water index, a repeatability study of Luminal water protocol was performed in 20 of the initial participants and 19 of the subsequent participants with 8-echo multiecho sequence using two different voxel size resolutions of 1.5×1.5×4 mm and 2×2×4 mm back-to-back. MRI parameters are listed in Table 2 below. One case was excluded as data was not useable due to the patient's movement during the scanning session.

TABLE 2

| Parameter | 8-echo |
|---|---|
| Number of echoes | 8 |
| TE (ms) | 31.25 |
| TR (ms) | 7,675 |
| Acquisition voxel size (mm³) | 2 × 2 × 4 |
| | 1.5 × 1.5 × 4 |
| FOV (mm³) | 180 × 180 × 68 |
| Scan Duration (mm:ss) | 02:56 |
| | 03:50 |

Sabouri, et al. [Ref 4, 5] used a regularized nonnegative least squares (NNLS) algorithm to fit a multiexponential model with a large number of exponentials to the signal decay curve. A 64-echo train length was used for multiecho spin-echo sequence. A large number of exponentials is computationally expensive. A 64-echo sequence is not usually available in a clinical scanner and requires complex set up. Devine, et al. [Ref 6] proposed a 32-echo acquisition as well as a simplified fitting model which uses only two Gaussian distributions to simulate the T₂ decay curve using a least squares regression. This fitting model minimizes the mean square error between actual signal and simulated signal over six parameters: M₀ (absolute signal magnitude), α (the magnitude ratio between two peaks), μ₁ (short T₂ peak), μ₂ (long T₂ peak), σ₁ (variance of short T₂ peak) and σ₂ (variance of long T₂ peak). μ₁ represents the compartment composed of stroma and epithelia which has shorter T₂ value and μ₂ represents the luminal compartment with longer T₂ values. Luminal water fraction (LWF) is then calculated as area of long T₂ peak/sum of area of long and short T₂ peak.

Previous work [Ref 7] demonstrated shorter echo train also have the ability to detect cancer. Experiments were conducted to assess the performance of Luminal Index (LI), which is derived by using first 8-echo T₂ data. LI is calculated as area of long T₂ peak divided by sum of area for short and long T₂ peak. Cancerous tissue has a T₂ value typically≤about 50 to 60 ms, and benign tissue usually has T₂ value≤about 2 s. By graphical observation, the majority short T₂ value is <200 ms. The values of μ₁ and μ₂ were constrained to be 0-200 ms and 200-2000 ms respectively. The ROI produced by radiologist earlier was superimposed onto the LI map and the median value of LI was calculated for each ROI. All data was processed using Matlab [R2019b 9.7.0.1190202].

Data were analyzed using PRISM [Version 8.3.0] to perform two comparisons. Firstly, a comparison between clinically significant (GL≥3+4) vs. nonsignificant (negative or GL=3+3) was performed to assess how well can LI differentiate lesions which need clinical attention and those which do not. A total of 113 lesions (82 from the initial study and 31 from the subsequent work) with Likert scores 3-5 and biopsy were analyzed using Mann-Whitney U test. The p value was less than 0.0001 and there was significant difference between the two groups.

Secondly, the mean values for sensitivity, specificity, and area-under-curve (AUC) values were also computed using a Receiver Operating Characteristic (ROC) analysis. A total of 142 cases (102 from initial patients and 40 from subsequent patients) with 29 (20 from initial patients and 9 from subsequent patients) Likert 2 cases included as a nonsignificant cancer group.

Prostate mpMRI studies are scored by radiologists a 1-5 scale of likelihood (Likert scale) of significant tumor (1=very unlikely, 2=unlikely, 3=equivocal, 4=likely, and 5=very likely). Patients with Likert scores of 1-2/5 throughout the prostate can safely avoid biopsy, whilst those with Likert scores of 4-5/5 undergo biopsy. FIG. 8 illustrates the Likert score and LI values for all Likert 3-4 biopsied patients. Significant differences in LI exist between biopsy positive and negative groups of patients scored Likert 3 (48 cases) those scored Likert 4 (34 cases) by radiologists. This suggests that using LI-MRI radiologists may better classify patients in the Likert 3 or 4 groups, avoiding unnecessary biopsy in those patients unlikely to have significant tumor.

Ten of the initial patients and 29 of the subsequent patients underwent two sequential LI-MRI scans within the same scanning session. The left and right TZ and left and right PZ for every slice were segmented by a single radiologist on the first scan. Regions were then transferred to the second scan. Adjustment was applied when there was significant displacement or patient movement between two scans. Bland-Altman analysis was performed to assess repeatability of measurements. The 95% limits of agreement are −80% to 78%.

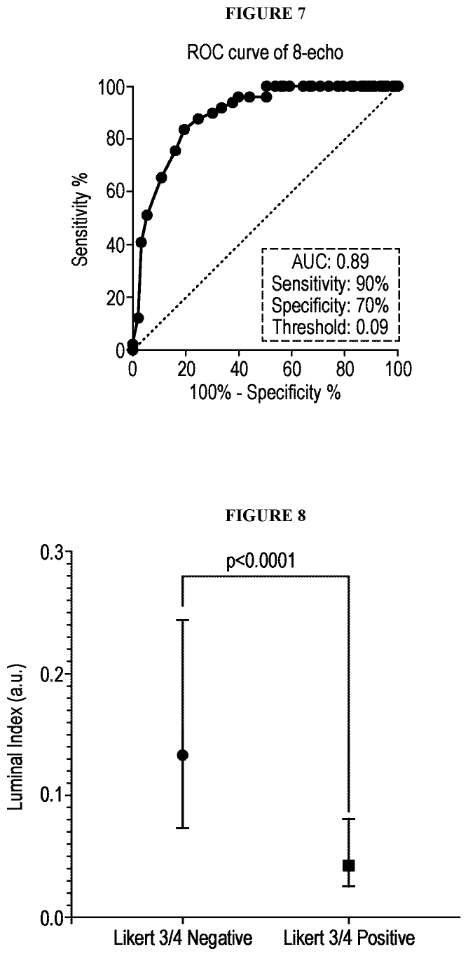

A threshold value of LI=0.09 (derived from ROC curve in FIG. 7) was chosen to achieve 90% sensitivity and 70% specificity. For comparison mpMRI has a sensitivity of approximately 90% and specificity of 50% for detection of significant prostate cancer [Ref 2].

It is desirable that peripheral zone (PZ) and transition zone (TZ) lesions are analyzed separately to obtain thresholds for color maps for each zone as the background normal zonal values differ. Separate thresholds can be derived empirically. Alternatively, it is possible to scale the TZ threshold based on the percentage difference between benign PZ and TZ regions. As an example, the threshold for TZ can be selected as ⅓ of the threshold for PZ.

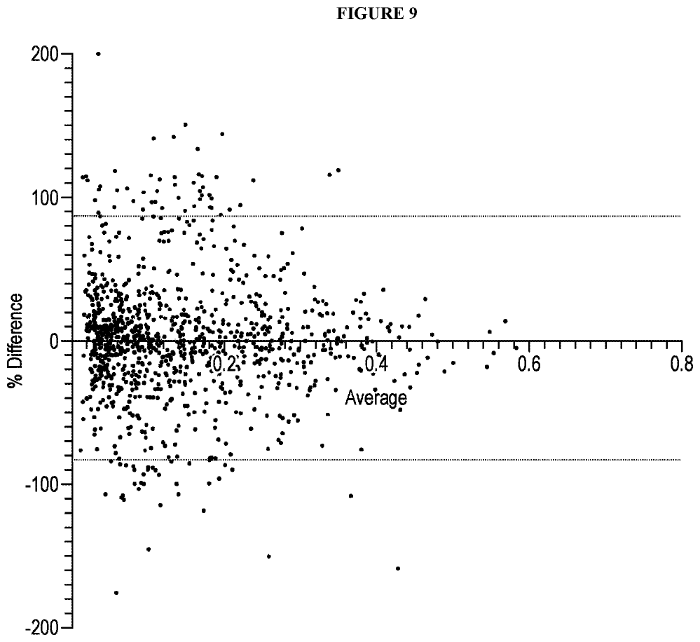

In order to account for measurement error, Bland—Altman 95% limits of agreement are used to determine the variation of the set thresholds (+78%/−80%). Bland-Altman analysis of LI values demonstrates a bias of −1.6% and 95% limits of agreement of −80% to 78% as shown in FIG. 9. This gives us an upper/lower bound for indeterminate pixels which then were assigned a yellow color. LI values which were less than the lower boundary were classified as malignant and marked with red. LI values greater than upper bound were classified as benign and marked with green.

For current processing of the LI map, a radiologist segments the PZ/TZ for each slice and a separate color map is generated for each zone. This is then combined to produce a single LI map. The process of PZ/TZ segmentation can be automated. In an embodiment, the LI map is presented to a user (e.g., a radiologist) in greyscale and the user is provided with separately adjustable filters for the PZ and TZ to enable the effects of different thresholds to be examined.

### CONCLUSION

The methods of the present invention may be performed by computer systems comprising one or more computers. A computer used to implement the invention may comprise one or more processors, including general purpose CPUs, graphical processing units (GPUs) or other specialized processors. A computer used to implement the invention may be physical or virtual. A computer used to implement the invention may be a server, a client, or a workstation. Multiple computers used to implement the invention may be distributed and interconnected via a local area network (LAN) or wide area network (WAN). Results of a method of the invention may be displayed to a user or stored in any suitable storage medium. The present invention may be embodied in a nontransitory computer-readable storage medium storing instructions to carry out a method of the invention. The present invention may be embodied in computer system comprising one or more processors and memory or storage storing instructions to carry out a method of the invention.

Having described exemplary embodiments of the invention, it will be understood that variations to the embodiment can be made within the scope of the invention, which is defined by the appended claims. For example, the invention may be applied to other glandular organs, such as pancreas, breast, etc. and to other animals.

### REFERENCES

1. Johnston, E. W., et al., “VERDICT MRI for Prostate Cancer: Intracellular Volume Fraction versus Apparent Diffusion Coefficient” Radiology, 2019, 291(2): pp. 391-397

2. Ahmed, H. U., et al., “Diagnostic accuracy of multi-parametric MRI and TRUS biopsy in prostate cancer (PROMIS): a paired validating confirmatory study” Lancet, 2017, 389(10071): pp. 815-822

3. Kasivisvanathan, V., et al., “MRI-Targeted or Standard Biopsy for Prostate-Cancer Diagnosis” N Engl J Med, 2018, 378(19): pp. 1767-1777

4. Sabouri, S., et al., “Luminal Water Imaging: A New MR Imaging T₂ Mapping Technique for Prostate Cancer Diagnosis” Radiology, 2017, 284(2): pp. 451-459

5. Sabouri, S., et al., “MR measurement of luminal water in prostate gland: Quantitative correlation between MRI and histology” J Magn Reson Imaging, 2017, 46(3): pp. 861-869

6. Devine, W., et al., “Simplified Luminal Water Imaging for the Detection of Prostate Cancer from Multiecho T₂ MR Images” J Magn Reson Imaging, 2019, 50(3): pp. 910-917

7. Gong, et al., “Optimisation of Luminal Water Imaging for Classification of Prostate Cancer” ISMRM 2019 Abstract #2371

What is claimed is:

1. An image processing method comprising:
receiving MRI data representing a scan of an organ of a patient, the MRI data including multiecho data for a plurality of pixels;
for each of a plurality of pixels of the MRI data:
fitting the multiecho data to a simulated decay curve;
calculating a tissue index based on at least one parameter of the simulated decay curve; and
comparing the tissue index to a threshold to determine a tissue type;
wherein each pixel of the multiecho data consists of 16 or fewer echoes.

2. A method according to claim 1 wherein each pixel of the multiecho data consists of 8 or fewer echoes, desirably 6 or fewer echoes.

3. A method according to claim 1 wherein the at least one parameter is selected from the group consisting of: area under long T₂ distribution (A_{L}), area under short T₂ distribution (A_{S}), Tₛₕₒᵣₜ, T_{long}, and the magnitude ratio between the long and short peaks (α).

4. A method according to claim 3 wherein calculating the tissue index comprises dividing the area under long T₂ distribution (A_{L}) of the simulated decay curve by the sum of the area under long T₂ distribution (A_{L}) and area under short T₂ distribution (A_{S}) of the simulated decay curve.

5. A method according to claim 4 wherein the threshold is in the range of from 0.05 to 0.15.

6. A method according to claim 1 wherein comparing the tissue index to a threshold comprises determining that a pixel likely corresponds to abnormal tissue if the tissue index is below a lower threshold and determining that a pixel likely corresponds to normal tissue if the tissue index is above an upper threshold.

7. A method according to claim 1 wherein comparing the tissue index to a threshold comprises comparing the tissue index corresponding to a first part of the organ to a first threshold and comparing the tissue index corresponding to a second part of the organ to a second threshold.

8. A method according to claim 1 wherein the fitting comprises determining a contour of an organ in the MRI data; determining median values of the multiecho data over the area of the organ; and setting the median values as initial parameters of a regression method.

9. A method according to claim 1 wherein the MRI data is a T₂ sequence.

10. A method according to claim 9 wherein fitting the multiecho data comprises fitting the multiecho data to a combination of a fast Gaussian distribution and a slow Gaussian distribution, the slow Gaussian distribution simulating a longer relaxation time than the fast Gaussian distribution.

11. A method according to claim 10 wherein calculating a tissue index comprises calculating a tissue index based on the areas under the fast Gaussian distribution and the slow Gaussian distribution.

12. A method according to claim 11 wherein calculating a tissue index comprises calculating a tissue index based on the area under a peak of the slow Gaussian distribution divided by the sum of the areas under a peak of the fast Gaussian distribution and the peak of the slow Gaussian distribution.

13. A computer program comprising executable code configured to perform a method comprising:
receiving MRI data representing a scan of an organ of a patient, the MRI data including multiecho data for a plurality of pixels:,
for each of a plurality of pixels of the MRI data:
fitting the multiecho data to a simulated decay curve;
calculating a tissue index based on at least one parameter of the simulated decay curve; and
comparing the tissue index to a threshold to determine a tissue type;
wherein each pixel of the multiecho data consists of 16 or fewer echoes.

14. A method of imaging comprising:
performing a magnetic resonance imaging process to obtain multiecho MRI data corresponding to a scan of an organ of a patient; and
processing the multiecho MRI data using a method comprising:
receiving MRI data representing a scan of an organ of a patient, the MRI data including multiecho data for a plurality of pixels;
for each of a plurality of pixels of the MRI data:
fitting the multiecho data to a simulated decay curve;
calculating a tissue index based on at least one parameter of the simulated decay curve; and
comparing the tissue index to a threshold to determine a tissue type;
wherein each pixel of the multiecho data consists of 16 or fewer echoes.

15. A method according to claim 14 wherein each pixel of the multiecho data consists of 8 or fewer echoes, desirably 6 or fewer echoes.

16. A computer program comprising executable code configured to control a magnetic resonance imaging apparatus to perform a scan of an organ of a patient and generate multiecho data consisting of 16 or fewer echoes, desirably 8 or fewer echoes, more desirably 6 or fewer echoes.

17. The method of claim 14 wherein the organ is selected from the group consisting of: prostate, pancreas, breast, and other glandular organs.
